(19) United States
(12) Patent Application Publication
(10) Pub. No.: US 2014/0315999 A1
(43) Pub. Date: Oct. 23, 2014

(54) USE OF PROLYL HYDROXYLASE INHIBITORS AS A RADIOPROTECTIVE DRUG FOR THE LOWER GASTROINTESTINAL TRACT

(71) Applicants: Amato Giaccia, Palo Alto, CA (US); Cullen Taniguchi, Palo Alto, CA (US)

(72) Inventors: Amato Giaccia, Palo Alto, CA (US); Cullen Taniguchi, Palo Alto, CA (US)

(21) Appl. No.: 14/240,968

(22) PCT Filed: Aug. 24, 2012
(86) PCT No.: PCT/US2012/052232

§ 371 Date: Jun. 2, 2014

(30) Foreign Application Priority Data
Aug. 26, 2011 (US) 61528076

(51) Int. Cl. A61K 31/225

(52) U.S. Cl. CPC A61K 31/225
USPC 514/547

(57) ABSTRACT

The present invention provides compositions and methods for treating or protecting mucosal tissues from damage associated with radiation and/or chemotherapy. Specifically, the instant invention is directed to inhibitors of prolyl hydroxylase domain (PHD) proteins and the use of such inhibitors in treating or protecting the gastrointestinal tract from damage associated with radiation.

### BACKGROUND

The present invention provides compositions and methods for protecting against toxicities associated with ionizing radiation, including radiotherapy, occupational exposure to radiation, and radiation encountered during military conflicts and terrorism.

Considering radiotherapy, many thousands of patients receive radiation therapy for cancer every year. Although in recent years radiation techniques have improved with regard to dosimetric accuracy, radiation toxicity remains a significant clinical problem resulting in treatment delays, increased patient hospitalization rates and remarkable short and long-term morbidity.

For example, external beam radiotherapy to the abdomen or pelvis is used in treating a variety of cancers, including those of the pancreas, cervix, rectum/anus, prostate and sarcoma. Such therapy is related to the development of radiation colitis, a consequence of radiation-induced mucosal and bowel wall injury.

The only approved radioprotector is amifostine, which must be administered systemically, and often causes dangerous hypotension. Thus, there remains a need for compounds and methods for protecting against radiation associated toxicities. The instant invention addresses this need and others.

### BRIEF DESCRIPTION OF THE DRAWINGS

FIG. 1 depicts a Western blot for HIF-1, HIF-2, and β-actin in colonic crypts at the time points indicated after DMOG injection (8 mg.) Each lane represents a different animal.

FIG. 2 is a graphical depiction of the crypts identified in transverse sections of colon stained with hematoxylin-eosin (H & E) after they were treated with saline or DMOG 16 hours before radiation, 4 hours before radiation, and then every 24 hours after receiving 20 Gy of abdominal radiation. The data represents the combined results from 8 mice receiving either DMOG or saline. Sixteen sections were analyzed per mouse. *p<0.006.

FIG. 3 is a graphical depiction of the crypts identified in transverse sections of colon stained with H & E in mice irradiated before administration of DMOG. Mice were treated with saline or DMOG 2 hours after receiving 20 Gy of abdominal radiation and then once every 24 hours thereafter for 4.5 days. x-axis, left bar mice treated with saline, right bar mice treated with DMOG; y-axis regenerated crypts. *p<0.005.

FIG. 4 depicts the concentration of FITC-Dextran present in the serum of mice gavaged with 0.6 mg./kg. FITC-Dextran (4 kD) either with or without irradiation and with or without DMOG. Six mice were tested per experimental condition. *p<0.04

FIG. 5 depicts Kaplan-Meier survival analysis of mice following whole-abdominal irradiation (20 Gy) with and without DMOG treatment. p=0.03 (n=12).

### BRIEF SUMMARY OF THE INVENTION

Gastrointestinal (GI) toxicity is one of the most common and distressing side effects of abdominopelvic radiation therapy. For instance, nearly 75% of patients receiving pelvic radiotherapy experience diarrhea, cramping, tenesmus and in rare instances hematochezia from rectal ulcers. The sensitivity of the normal cells in the GI tract limits dose escalation of radiotherapy to tumors in the abdomen or pelvis, which can cause morbidity even when highly conformal radiation techniques are employed. Unfortunately, there are no effective treatments to prevent these GI side effects.

The present invention provides methods for alleviating the side effects associated with the damaging effects of radiation. This novel method involves administering an agent or agents that inhibit prolyl hydroxylases domain (PHD) protein activity.

According, in some embodiments is provided a method for alleviating the effects of radiation. This method entails administering to subject an effective amount of a prolyl hydroxylase domain (PHD) inhibitor. Such PHD inhibitors encompass compounds with the formula I

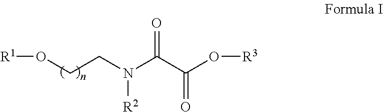

in which R¹ and R³ are independently members selected from substituted or unsubstituted C₆ alkyl; R² is selected from H and substituted or unsubstituted C₁-C₆ alkyl and n is an integer from 1 to 10.

In some embodiments, a method of alleviating proctopathy associated with radiation are provided, the provided methods encompass administering an effective amount of a PHD inhibitor such as the one described above. In other embodiments, the PHD inhibitor is Dimethyloxallyl Glycine (DMOG; N-(methoxyoxoacetyl)-glycine methyl ester).

In other embodiments, a method of alleviating the affects of abdominopelvic radiation therapy are provided, the provided methods encompass administering an effective amount of a PHD inhibitor such as one with the generic formula, Formula I. In other embodiments, the PHD inhibitor is DMOG.

Other advantages, aspects and objects of the invention are apparent from the detailed description which follows.

### DETAILED DESCRIPTION OF THE INVENTION

### Introduction

Inhibitors of prolyl hydroxylases domain (PHD) proteins have been considered in treating a wide range of maladies including anemia, cardiovascular and neural ischemia, metabolic disorders, such as obesity, and enhanced wound healing. Disclosed herein is the remarkable finding that PHD inhibitors, for example DMOG, are useful in alleviating the effects of radiation exposure and systemic chemotherapeutic agents, especially the effects on the gastrointestinal tract.

Prolyl hydroxylases domain (PHD) proteins act as cellular oxygen sensors. Under conditions of normal oxygen tension, PHD proteins use molecular oxygen, along with co-factors iron and 2-oxoglutarate, to drive hydroxylation of prolines 402 and 564 of hypoxia-inducible transcription factor (HIF). These hydroxyproline moieties serve as docking sites for the von Hippel Lindau (VHL) protein, an E3 ubiquitin ligase, which targets HIF for proteasomal degradation. Thus, under normoxia, HIF is marked for ubiquitinization and subsequent proteasome-dependent degradation.

Alternatively, in situations in which the oxygen supply to cells becomes inadequate, prolyl hydroxylase activity is inhibited, HIF is not hydroxylated, and therefore escapes VHL-dependent degradation. Consequently, sufficient HIF subunits are enabled to translocate to the nucleus, where they associate with HIF-1β (also referred to as Aryl Hydrocarbon Receptor Nuclear Translocator, ARNT).

In the nucleus, heterodimers of HIF bind to DNA-specific hypoxia response elements of different target genes and thereby activating several biological pathways which act as a means for reducing the affects of hypoxia; this includes the transcription of genes implicated in the control of metabolism and angiogenesis as well as apoptosis and cellular stress.

The instant disclosure provides that inhibitors of PHD proteins effectively radioprotects the colon from relatively high doses of abdominal radiation. Accordingly, inhibitors of PHD can be used to ameliorate unwanted radiation damage to the colorectal region during pelvic and/or prostate radiotherapy, thereby becoming a regular complement to radiation therapy treatments of the abdomen and pelvis.

It is believed that the present invention will be better understood from the following definitions.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures of analytical and synthetic organic chemistry described below are those well known and commonly employed in the art. Standard techniques, or modifications thereof, are used for chemical syntheses and chemical analyses.

“Alleviate” and grammatical equivalents refers to, for example, a detectable improvement or a detectable change consistent with improvement that occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range between about any two of these values. Such improvement or change may be observed in treated subjects as compared to subjects not treated with a PHD inhibitor, where the untreated subjects have, or are subject to developing, the same or similar disease, condition, and/or symptom. Amelioration of a disease, condition, symptom or assay parameter may be determined subjectively or objectively, e.g., self assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s) or, for example, by detection of enteritis or diarrhea within a subject. Alleviation may be transient, prolonged or permanent or it may be variable at relevant times during or after a PHD inhibitor is administered to a subject or is used in an assay or other method described herein or a cited reference, e.g., within timeframes described below.

“Therapeutically effective amount” refers to the amount of a therapy (e.g., a composition containing a PHD inhibitor), which is sufficient to reduce the severity of the effects of radiation exposure; for example, such effects may be enteritis, diarrhea, cramping, tenesmus, ulcer formation and/or hematochezia, reduce the duration of enteritis, diarrhea, cramping, tenesmus, and ulcers, prevent the advancement of enteritis, diarrhea, cramping, tenesmus, and ulcers, cause regression of enteritis, diarrhea, cramping, tenesmus, ameliorate one or more symptoms associated with enteritis, diarrhea, cramping, tenesmus, ulcer formation or enhance, facilitate, or improve the therapeutic effect(s) of another therapy.

As used herein, the terms “prevent,” “preventing,” and “prevention” refer to the prevention of the recurrence, onset, or development of or one or more symptoms thereof in a subject resulting from exposure to radiation. For example, the administration of an abdominopelvic therapy. Preventing includes protecting against radiation induced enteritis, protecting against radiation induced injury to the mucosa of the colon, protecting against radiation induced colorectal inflammation, and/or radiation-induced inflammation or bacterial invasion of other portions of the alimentary tract. For example, the PHD inhibitor may be provided as an intraperitoneal injection to treat or ameliorate radiation-induced enteritis, diarrhea, cramping, tenesmus, and/or hematochezia or other radiation-induced mucositis and ulcer formation.

As used herein, the terms “treat,” “treatment,” and “treating” refer to the reduction of the progression, severity, and/or duration of enteritis, diarrhea, cramping, tenesmus, and/or hematochezia or amelioration of one or more symptoms thereof, wherein such reduction and/or amelioration result from the administration of one or more therapies (e.g., a composition containing a PHD inhibitor such as DMOG

As used herein the terms “radiation,” “radiation therapy,” “radiotherapy,” and “irradiation” refer to any exposure to ionizing radiation whether intentional or unintentional, malicious or therapeutic, and may include, for example, external beam radiotherapy, photon radiotherapy, electron radiotherapy, proton radiotherapy, carbon ion radiotherapy, lithium ion radiotherapy, silicon ion radiotherapy, helium ion radiotherapy, other forms of hadrontherapy or other particle therapy, brachytherapy, radioisotope therapy, injectable isotopes, e.g., isotopes adhered to or within or admixed with a matrix of any sort, or any radiation exposure that is unintentional or malicious, independent of the agent or agents employed.

“Ionizing radiation” refers to radiation that has sufficient energy to eject one or more orbital electrons from an atom or molecule (e.g. α-particles, β-particles, γ-rays, x-rays, neutrons, protons, and other particles having sufficient energy to produce ion pairs in matter.

“Radiation dose” refers to the total amount of radiation absorbed by material or tissues. Radiation dose is measured in several different units, but all relate to the amount of energy deposited. The units include the roentgen (R), the gray (Gy), and the sievert (Sv). The sievert and gray are similar, except the sievert takes into account the biologic effects of different types of radiation.

“Radiation dose rate” refers to the radiation dose (dosage) absorbed per unit of time.

The term “LDx/y” refers to the average dose of radiation which results in death of x % of subjects by y days. For example, the terms LD50/30 and LD50/60 refer to the average dose of radiation which results in death of 50% of the subjects by 30 or 60 days, respectively.

As used herein, “administering” means, for example, intraperitoneal administration, administration as a suppository, enema, topical contact, intravenous, intramuscular, intralesional, or subcutaneous administration, administration by inhalation, or the implantation of a slow-release device, e.g., a mini-osmotic pump, to the subject. Administration is by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

As used herein, the terms “subject” and “subjects” refers to an animal, preferably a mammal, including a non-primate (e.g., a cow, pig, horse, cat, or dog), a primate (e.g., a monkey, chimpanzee, or human), and more preferably a human. In a some embodiments, the subject is a mammal, preferably a human, who has been exposed to or is going to be exposed to an insult that may induce enteritis, diarrhea, cramping, tenesmus, rectal ulcers and/or hematochezia (such as ionizing radiation, for example in the form of radiation therapy). In another embodiment, the subject is a farm animal (e.g., a horse, pig, or cow) or a pet (e.g., a dog or cat) that has been exposed to or is going to be exposed to a similar insult.

“Inhibit” and grammatical equivalents refer to reducing or blocking the activity of prolyl hydroxlase domain proteins. For example, by reducing the capacity of PHD proteins to hydroxlate a target proline, such as prolines 402 and 564 of HIF. Inhibition can be expressed in terms of percentage and/or fold; such as 1%, 5%, 10%, 20%, 30%, 40%, 50%, 60%, 70%, 80%, 90%, 95%, 99% reduction and/or a 2-fold reduction, 5-fold reduction, 10-fold reduction, 20-fold reduction, 50-fold reduction, 100-fold reduction, 500-reduction, 1000-fold reduction or more and ranges in between.

The term “alkyl”, by itself or as part of another substituent, means a straight or branched chain hydrocarbon radical, which may be fully saturated, mono- or polyunsaturated. For convenience, the term alkyl may refer to divalent (i.e., alkylene) and other multivalent radicals in addition to monovalent radicals. Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl and the like. An unsaturated alkyl group is one having one or more double bonds or triple bonds (i.e., alkenyl and alkynyl moieties). Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

Typically, an alkyl (or alkylene) group will have from 1 to 30 carbon atoms, That is, in some embodiments, alkyl refers to an alkyl having a number of carbons selected from C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀ and any combination thereof. In some embodiments, alkyl refers to C₁-C₂₅ alkyl. In some embodiments, alkyl refers to C₁-C₂₀ alkyl. In some embodiments, alkyl refers to C₁-C₁₅ alkyl. In some embodiments, alkyl refers to C₁-C₁₀ alkyl. In some embodiments, alkyl refers to C₁-C₆ alkyl.

The term “heteroalkyl”, by itself or in combination with another term, means an alkyl in which at least one carbon is replaced with an atom other than carbon (i.e., a heteroatom). In some embodiments, the heteroatom is selected from O, N and S, wherein the nitrogen and sulfur atoms may optionally be oxidized and the nitrogen heteroatom may optionally be quaternized. In some embodiments, a heteroalkyl is any C₂-C₃₀ alkyl, C₂-C₂₅ alkyl, C₂-C₂₀ alkyl, C₂-C₁₅ alkyl, C₂-C₁₀ alkyl or C₂-C₆ alkyl in any of which one or more carbons are replaced by one or more heteroatoms selected from O, N and S. The heteroatoms O, N and S may be placed at any interior position of the heteroalkyl group and may also be the position at which the heteroalkyl group is attached to the remainder of the molecule. In some embodiments, depending on whether a heteroatom terminates a chain or is in an interior position, the heteroatom may be bonded to one or more H or C₁, C₂, C₃, C₄, C₅ or C₆ alkyl according to the valence of the heteroatom. Examples include —CH₂—CH₂—O—CH₃, —CH₂—CH₂—NH—CH₃, —CH₂—CH₂—N(CH₂)—CH₃, —CH₂—S—CH₂—CH₃, —CH₂—CH₂, —S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃. The term “heteroalkylene” may be use to refer a divalent radical derived from heteroalkyl. Unless otherwise stated, no orientation of the linking group is implied by the direction in which a divalent group is written. For example, the formula —C(O)₂R′ represents both —C(O)₂R′ and R′C(O)₂.

The terms “cycloalkyl” and “heterocycloalkyl”, by themselves or in combination with other terms refer to cyclic versions of “alkyl” and “heteroalkyl”, respectively. For heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1 (1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1 piperazinyl, 2-piperazinyl and the like.

The terms “halo” or “halogen” refer to fluorine, chlorine, bromine and iodine. Additionally, terms such as “haloalkyl,” are meant to include monohaloalkyl and polyhaloalkyl.

The term “aryl” refers to a polyunsaturated, aromatic hydrocarbon that can be a single ring or multiple rings (preferably 1, 2 or 3 rings) that are fused together or linked covalently. For convenience, the term aryl may refer to divalent (i.e., arylene) and other multivalent radicals in addition to monovalent radicals. In some embodiments, aryl is a 3, 4, 5, 6, 7 or 8 membered ring that is optionally fused to one or two other 3, 4, 5, 6, 7 or 8 membered rings.

The term “heteroaryl” refers to aryl containing 1, 2, 3 or 4 heteroatoms selected from N, O and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl and 6-quinolyl.

In some embodiments, any alkyl, heteroalkyl, cycloalkyl, heterocycloalkyl, aryl and heteroaryl may be substituted. Preferred substituents for each type of radical are provided below.

Substituents for alkyl, heteroalkyl, cycloalkyl and heterocycloalkyl radicals (including those groups often referred to as alkylene, alkenyl, heteroalkylene, heteroalkenyl, alkynyl, cycloalkenyl, and heterocycloalkenyl) are generically referred to as “alkyl group substituents”. In some embodiments, an alkyl group substituent is selected from R′, —OR′, ═O, ═NR′, ═N—OR′, —NR′R″, —SR′, -halogen, SiR′R″R′″, OC(O)R′, —C(O)R′, —CO₂R′, —CONR′R″, —OC(O)NR′R″, —NR″C(O)R′, NR′C(O)NR″R′″, —NR″C(O)₂R′, —NR—C(NR′R″R′″)═NR″″′, NR′C(NR′R″)═NR″′, S(O)R′, —S(O)₂R′, —S(O)₂NR′R″, NRSO2R′, —CN and NO₂. Embodiments of R′, R″, R″′ and R″″ are provided below. Substituents for aryl and heteroaryl groups are generically referred to as “aryl group substituents”. In some embodiments, an aryl group substituent is selected from R′, OR′, ═O, ═NR′, ═NOR′, —NR′R″, —SR′, halogen, —SiR′R″R″′, OC(O)R′, —C(O)R′, CO₂R′, CONR′R″, —OC(O)NR′R″, NR″C(O)R′, NR′C(O)NR″R′″, —NR″C(O)₂R′, NRC(NR′R″R″′)═NR″″′, NRC(NR′R″)═NR″′, —S(O)R′, —S(O)₂R′, S(O)₂NR′R″, NRSO2R′, —CN, NO₂ and N₃. In some embodiments, R′, R″, R″′ and R″″ are each independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. In some embodiments, R′, R″, R″′ and R″″ are each independently selected from hydrogen, unsubstituted alkyl, unsubstituted heteroalkyl, unsubstituted cycloalkyl, unsubstituted heterocycloalkyl, unsubstituted aryl and unsubstituted heteroaryl. In some embodiments, R′, R″, R″′ and R″″ are each independently selected from hydrogen and unsubstituted alkyl (e.g., C₁, C₂, C₃, C₄, C₅ and C₆ alkyl).

Two substituents on adjacent atoms of an aryl or heteroaryl ring may optionally be replaced with a substituent of the formula T-C(O)—(CRR′)q-U—, wherein T and U are independently selected from NR—, —O—, —CRR′— and a single bond, and q is an integer selected from 0, 1, 2 and 3. Alternatively, two of the substituents on adjacent atoms of an aryl or heteroaryl ring may optionally be replaced with a substituent of the formula A (CH₂)r B—, wherein A and B are independently selected from CRR′—, —O—, —NR—, —S—, S(O)—, S(O)₂—, S(O)₂NR′ and a single bond, and r is an integer selected from 1, 2, 3 and 4. One of the single bonds of the new ring so formed may optionally be replaced with a double bond. Alternatively, two of the substituents on adjacent atoms of the aryl or heteroaryl ring may optionally be replaced with a substituent of the formula (CRR′)s X (CR″R″′)d, where s and d are independently integers selected from 0, 1, 2 and 3, and X is selected from O—, NR′—, —S—, —S(O)—, —S(O)₂— and S(O)₂NR′—. The substituents R, R′, R″ and R″′ are preferably independently selected from hydrogen and substituted or unsubstituted (C₁-C₆)alkyl.

Unless otherwise specified, the symbol “R” is a general abbreviation that represents a substituent group that is selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound includes more than one R, R′, R″, R″′ and R″″ group, they are each independently selected.

For groups with exchangeable or acidic protons, the ionized form is equally contemplated. For example, COOH also refers to COO— while SO₃H also refers to SO³⁻.

For the purposes of the present disclosure the terms “compound,” “analog,” and “composition of matter” stand equally well for the PDH enzyme inhibitors described herein, including all enantiomeric forms, diastereomeric forms, salts, and the like, and the terms “compound,” “analog,” and “composition of matter” are used interchangeably throughout the present specification.

### The Biological Effects of Radiation Exposure

Radiation exposure can result from radiation therapy, accidental exposure, for example in an occupational setting, and radiation exposure from military action or a terrorist attack. See e.g., Moulder, Int. J. Radiat. Biol. 80:3-10 (2004).

The major biological effects of radiation exposure are the destruction of bone marrow cells, gastrointestinal (GI) damage, lung pneumonitis, and central nervous system (CNS) damage.

Accordingly, in some embodiments, methods and compositions are provided for alleviating bone marrow toxicity associated with ionizing radiation, wherein the methods entail the use, and the compositions are, PHD protein inhibitors. In other embodiments, methods and compositions are provided for alleviating GI toxicity associated with ionizing radiation, wherein the methods entail the use, and the compositions are, PHD protein inhibitors. In some embodiments, methods and compositions are provided for alleviating esophageal toxicity associated with ionizing radiation, wherein the methods entail the use, and the compositions are, PHD protein inhibitors.

The damaging effects of radiation depend on several factors, including the amount (dose) and duration of exposure. A single, rapid dose of radiation to the entire body can be fatal, but the same total dose given over a period of weeks or months may have much less effect. For a given dose, genetic damage is more likely with rapid exposure. The effects of radiation also depend on how much of the body is exposed. For example, more than 6 Gy generally causes death when the radiation is distributed over the entire body due to effects on the hematopoietic system; however, when concentrated in a small area, as in radiation therapy for cancer, three or four times this amount can be given without serious harm to the subject as a whole.

Radiation exposure produces two types of injury: acute (immediate) and chronic (delayed). Acute radiation injury triggers inflammation through vascular endothelial damage leading to leaking vessels. A vascular response and a cellular response follow. Ionizing radiation depresses immunity and damages intestinal epithelium, both of which promote microbial translocation from the intestines.

It has been estimated that the exposure of 100 rems (roentgen equivalent man: a measurement used to quantify the amount of radiation that would produce harmful biological effects) would produce acute radiation syndrome symptoms. Exposure levels above 300 rems would result in the death of approximately 50% of the exposed population.

Accordingly, in some embodiments, methods and compositions for alleviating the toxic effects associated with lethal radiation exposure are provided, wherein the methods entail the use of, and the compositions are, PHD inhibitors and wherein a lethal dose is a dose sufficient to result in the death or 50% or more of the population (cells or animals) exposed to that dose.

Subjects may be exposed to ionizing radiation when undergoing therapeutic irradiation for the treatment of proliferative disorders. Such disorders include cancerous and non-cancer proliferative disorders. Formulations described herein are effective in protecting normal cells during therapeutic irradiation of a broad range of tumor types, including but not limited to the following: breast, prostate, ovarian, lung, colorectal, brain (i.e., glioma) and renal.

Radiation therapy for cancer mainly produces symptoms in the part of the body that receives radiation. For example, in radiation therapy for rectal cancer, abdominal cramping and diarrhea are common because of the effects of radiation on the small intestine.

Thus, in some embodiments are provided methods and compositions for alleviating the affects of abdominopelvic radiation therapy, wherein the methods entail the use of, and the compositions are, a PHD protein inhibitor.

In other embodiments, a method for alleviating gastrointestinal toxicity associated with radiation therapy is provided, the method entailing the administration of a PHD protein inhibitor.

In some embodiments a method for alleviating the affects of mucositis are provided wherein mucositis refers to inflammation of mucosal membranes induced by radiation or chemotherapy. The term “acute mucositis,” as used herein means mucositis occurring within 90 days of treatment initiation, characterized by parched or confluent pseudomembranes, ulceration, or necrosis. The term “late mucositis,” as used herein means mucositis: occurring 90 days to 2 years after treatment initiation, characterized by mucosal atrophy, dryness, telangiectasia or ulceration.

The disclosed methods further provide a useful means for administering a PHD inhibitor to subjects whose bone marrow has been exposed to radiation. Thus, in some embodiments, methods and compositions are provided for alleviating bone marrow toxicity associated with radiotherapy are provided, wherein the methods entail the use, and the compositions are, PHD protein inhibitors.

In some embodiments, methods and compositions for alleviating toxicity associated with radiation therapy directed to the head and neck are provided, wherein the methods make use of, and the compositions are, PHD protein inhibitors. In some embodiments, methods and compositions for alleviating radiation therapy associated xerostomia are provided, wherein the methods make use of, and the compositions are, PHD inhibitors.

“The term “xerostomia,” as used herein means dryness of mouth due to salivary gland dysfunction induced by radiation. The term “acute xerostomia,” as used herein means xerostomia occurring within 90 days of treatment initiation, characterized by dryness of mouth with thick, sticky saliva, altered taste, or acute salivary gland necrosis. The term “late xerostomia,” as used herein means xerostomia occurring 90 days to 2 years after treatment initiation, characterized by dryness, poor saliva production, or fibrosis of salivary glands.

### Prolyl Hydroxylase Domain (PHD) Proteins

Prolyl hydroxylase domain (PHD) proteins, also referred to as HIF-prolyl hydroxylases, form an evolutionarily conserved subfamily of dioxygenases. So far, at least four members belonging to this subfamily have been identified in mammals including PHD1, as referred to as EGLN2/HPH3 (GenBank Accession No. CAC42511), PHD2, also referred to as EGLN1/HPH2 (GenBank Accession No. AAG33965; Dupuy et al. (2000) Genomics 69:348-54), and PHD3, also referred to as EGLN3/HPH1 (GenBank Accession No. CAC42517)

PHD genes were originally labeled EGLN (1, 2, 3) for Egg-Laying Mutant Nine (derived from C. elegans gene name) which the protein was referred to as prolyl hydroxylase domain (PHD 2, 1, 3) or HIF-Prolyl Hydroxylase (HPH 1, 2, 3). Additionally, while the names of the proteins were interchangeable, the gene identifier (i.e., the notation of “1” “2” and “3”) has not been consistently employed in the literature. For example, HPH 1, 2, 3 equate with PHD 2, 1 and 3 respectively, and with EGLN 1, 2, and 3, respectively. However, in other instances the literature did not follow this convention.

In addition to serving as oxygen sensors, PHDs may serve other biological functions as well.

### Pharmaceutical Compositions

The instant invention provides compositions for alleviating the effects of ionizing radiation exposure in a subject, the methods encompassing the administration of PHD inhibitors with the generic structure as represented below by Formula I

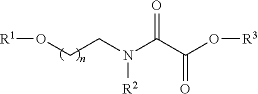

wherein R¹ and R³ are independently members selected from substituted or unsubstituted C₁-C₆ alkyl; R² is selected from H and substituted or unsubstituted C₁-C₆ alkyl and n is an integer selected from 0, 1, 2, 3, 4, 5, 6, 7, 8, 9, or 10.

In other embodiments, the PHD inhibitor is Dimethyloxallyl Glycine (DMOG; N-(methoxyoxoacetyl)-glycine methyl ester).

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Greene and Wuts (1991), and references cited therein.

Furthermore, the compounds of this invention will typically contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

### Modes of Administration and Pharmaceutical Compositions

In some embodiments, methods and compositions are provided for alleviating the effects of exposure of a subject to ionizing radiation are provided, wherein the methods use, and the compositions are, PHD inhibitor(s), are administered to the subject prior to ionizing radiation exposure. In other embodiments, the PHD inhibitor(s) are administered at the same time as ionizing radiation exposure. In other embodiments, the PHD inhibitor(s) are administered after ionizing radiation exposure. In some embodiments, the ionizing radiation is radiotherapy.

The routes of administration of a compound of the present invention will vary, naturally, with the location and nature of the condition to be treated, and include, e.g., inhalation, intradermal, transdermal, parenteral, intravenous, intramuscular, intranasal, subcutaneous, percutaneous, intratracheal, intraperitoneal, intratumoral, perfusion, lavage, direct injection, and oral administration and formulation. As detailed below, PHD inhibitors can be administered as injectable liquids by intravascular, intravenous, intra-arterial, intracerobroventicular, intraperitoneal, subcutaneous administration, as topical liquids or gels, or in solid oral dosage forms.

The amounts may vary depending on the type of biological matter (cell type, tissue type, organism genus and species, etc.) and/or its size (weight, surface area, etc.).

In some embodiments, methods and compositions are provided for alleviating the effects of exposure of a subject to ionizing radiation are provided, wherein the subject is a mammal. In other embodiments, the subject is a mammal but not a human. In some embodiments the subject is a human.

It will generally be the case that the larger the organism, the larger the dose. Therefore, an effective amount for a mouse will generally be lower than an effective amount for a rat, which will generally be lower than an effective amount for a dog, which will generally be lower than an effective amount for a human. The effective concentration of a compound of the present invention depends on the dosage form and route of administration. For intravenous administration, in some embodiments effective concentrations are in the range of 0.5 to 50 milligrams per kilogram of body weight delivered continuously.

Similarly, the length of time of administration may vary depending on the type of biological matter (cell type, tissue type, organism genus and species, etc.) and/or its size (weight, surface area, etc.) and will depend in part upon dosage form and route of administration. In particular embodiments, a compound of the present invention may be provided for about or at least 30 seconds, 1 minute, 2 minutes, 3 minutes, 5 minutes, 10 minutes, 15 minutes, 30 minutes, 1 hour, 2 hours, 3 hours, four hours five hours, six hours, eight hours, twelve hours, twenty-four hours, or greater than twenty-four hours. A compound of the present invention may be administered in a single dos or multiple doses, with varying amounts of time between administered doses.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Alternatively, the amount specified may be the amount administered as the average daily, average weekly, or average monthly dose.

The PHD inhibitor can be administered to a subject in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg or more, or any range derivable therein.

Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to, for example, subject surface area).

Concentrations of the PHD inhibitor can be in doses of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 or more mg or mg/m² (with respect to, for example, subject surface area), or any range derivable therein. Alternatively, the amount specified may be the amount administered as the average daily, average weekly, or average monthly dose, or it may be expressed in terms of mg/kg, where kg refers to the weight of the patient and the mg is specified above.

### Injectable Compositions and Formulations

In some embodiments, route of administering compositions for alleviating the effects of ionizing radiation exposure are provided, wherein the compositions are PHD inhibitors and the route of administration is intraperitoneal.

In other embodiments, the methods for the delivery of the PHD inhibitor of the present invention may comprise intravenous injection, perfusion of a particular area, and/or oral administration. However, the pharmaceutical compositions disclosed herein may alternatively be administered parenterally, rectally, intradermally, intramuscularly, transdermally or even intraperitoneally.

Injection of the PHD inhibitor may be delivered by syringe or any other method used for injection of a solution, as long as the solution can pass through the particular gauge of needle required for injection. A novel needleless injection system has recently been described (U.S. Pat. No. 5,846,233, incorporated herein by reference in its entirety) having a nozzle defining an ampule chamber for holding the solution and an energy device for pushing the solution out of the nozzle to the site of delivery.

Solutions of the PHD inhibitor may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, incorporated herein by reference in its entirety).

The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580).

Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the PHD inhibitors in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are typically vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

PHD inhibitors and their related compositions as described herein may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

In certain formulations, the PHD inhibitor is formulated as a dry power. It is a further object of the present invention to use, for the process, readily accessible cheap raw materials in the form of dairy by-products, in place of pure carbohydrates.

The PHD inhibitors are administered in a manner compatible with the dosage formulation, and in such amount as will be therapeutically effective. The quantity to be administered depends on the subject and condition to be treated, including, e.g., the area, organ, tissue, etc. to be subject to radiation. Precise amounts of the PHD inhibitor required to be administered depend on the judgment of the practitioner.

Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

The manner of application may be varied widely. Any of the conventional methods for administration are applicable. These are believed to include oral application on a solid physiologically acceptable base or in a physiologically acceptable dispersion, parenterally, by injection or the like. The dosage will depend on the route of administration and will vary according to the size of the host.

In many instances, it will be desirable to have multiple administrations of one or more the PHD inhibitors.

### Bolus Administration

Bolus administration generally refers to a single dose comprising one or more Effective Compounds. The term “bolus” is intended to exclude dosage forms such as sustained release, pulsed release, and time release, and includes any dosage form which can be used to deliver a single dose. Typically, bolus administration takes place intravenously by direct infusion, injection or gravity drip but other means, such as oral dosage forms, are also envisioned. In some embodiments, a bolus administration may comprise a single dose of a concentrated form of one or more Effective Compounds given over a period of time. In some embodiments, a bolus administration may comprise a single large dose given over a short period of time. A bolus may be administered to a patient in need of treatment once daily, such as in the morning. The bolus dosages of the present invention may be administered in any conventional form known to those of skill in the art.

### Topical Formulations and Uses Thereof

Radiation therapy and chemotherapy for the treatment of cancer damage normal cells in the oral mucosa, leading to the unintended, but debilitating side effects of therapy, oral mucositis. In some embodiments, methods for slowing, limiting or preventing damage to cells lining the mouth, esophagus and tongue and the resultant painful condition of oral mucositis are provided.

In certain embodiments the PHD inhibitor is administered topically. This is achieved by formulating the PHD inhibitor in a cream, gel, paste, or mouthwash and applying such formulation directly to the areas that require exposure to the PHD inhibitor (e.g., mouth, tongue, throat).

The topical compositions of this invention can be formulated as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762, each of which is incorporated herein by reference in its entirety.

Possible pharmaceutical preparations that can be used rectally include, for example, suppositories, which consist of a combination of one or more of the Effective Compounds with a suppository base. Suitable suppository bases are, for example, natural or synthetic triglycerides, or paraffin hydrocarbons. In addition, it is also possible to use gelatin rectal capsules which consist of a combination of the Effective Compounds with a base. Possible base materials include, for example, liquid triglycerides, polyethylene glycols, or paraffin hydrocarbons.

### Perfusion Systems

A perfusion system for cells may be used to expose a tissue or organ to the PHD inhibitor in the form of a liquid or a semi-solid. Perfusion refers to continuous flow of a solution through or over a population of cells. It implies the retention of the cells within the culture unit as opposed to continuous-flow culture, which washes the cells out with the withdrawn media (e.g., chemostat). Perfusion allows for better control of the culture environment (pH, pO₂, nutrient levels, PHD inhibitor levels, etc.) and is a means of significantly increasing the utilization of the surface area within a culture for cell attachment.

The technique of perfusion was developed to mimic the cells milieu in vivo where cells are continuously supplied with blood, lymph, or other body fluids. Without perfusion of a physiological nutrient solution, cells in culture go through alternating phases of being fed and starved, thus limiting full expression of their growth and metabolic potential. In the context of the present invention, a perfusion system may also be used to perfuse cells with the PHD inhibitor.

Those of skill in the art are familiar with perfusion systems, and there are a number of perfusion systems available commercially. Any of these perfusion systems may be employed in the present invention. One example of a perfusion system is a perfused packed-bed reactor using a bed matrix of a non-woven fabric (CelliGen™, New Brunswick Scientific, Edison, N.J.; Wang et al., 1992; Wang et al., 1993; Wang et al., 1994). [1620] J. Other Apparatuses

### Further Delivery Devices or Apparatuses

In some embodiments it is contemplated that methods or compositions will involve a specific delivery device or apparatus. Any method discussed herein can be implemented with any device for delivery or administration including, but not limited, to those discussed herein.

For topical administration of the PHD inhibitor of the invention may be formulated as solutions, gels, ointments, creams, suspensions, etc. as are well-known in the art. Systemic formulations may include those designed for administration by injection or infusion, e.g., subcutaneous, intravenous, intramuscular, intrathecal or intraperitoneal injection, as well as those designed for transdermal, transmucosal, oral or pulmonary administration.

For oral administration, the PHD inhibitor of the invention to be formulated as tablets, pills, dragees, capsules, liquids, gels, syrups, slurries, suspensions and the like, for oral ingestion by a patient to be treated or oral liquid preparations such as, for example, suspensions, elixirs and solutions.

For buccal administration, the compositions may take the form of tablets, lozenges, etc. formulated in conventional manner. Other intramucosal delivery might be by suppository or intranasally.

For administration directly to the lung by inhalation the compound of invention may be conveniently delivered to the lung by a number of different devices.

Transdermal administration of the compound of the invention can be achieved by medicated device or patch which is affixed to the skin of a patient. The patch allows a medicinal compound contained within the patch to be absorbed through the skin layers and into the patient's blood stream.

### Combination Therapies

The compounds and methods of the present invention may be used in the context of a number of therapeutic and diagnostic applications. In order to increase the effectiveness of a treatment with the compositions of the present invention, it may be desirable to combine PHD inhibitor compositions with other agents effective in the treatment of those diseases and conditions (secondary therapy). For example, the treatment of stroke (antistroke treatment) typically involves an antiplatelet (aspirin, clopidogrel, dipyridamole, ticlopidine), an anticoagulant (heparin, warfarin), or a thrombolytic (tissue plasminogen activator).

Various combinations may be employed; for example, a PHD inhibitor, such as DMOG, and a secondary therapy.

Administration of the PHD inhibitors of the present invention to biological matter will follow general protocols for the administration of that particular secondary therapy, taking into account the toxicity, if any, of a PHD inhibitor treatment. It is expected that the treatment cycles would be repeated as necessary. It also is contemplated that various standard therapies, as well as surgical intervention, may be applied in combination with the described therapies.

The following examples are provided by way of illustration only and are not meant to limit the scope of the invention. Those of skill in the art will readily recognize a variety of non-critical parameters that could be changed or modified to yield essentially similar results.

### EXAMPLES

### Example 1

### The Effects of DMOG on HIF Protein Levels at Various Timepoints

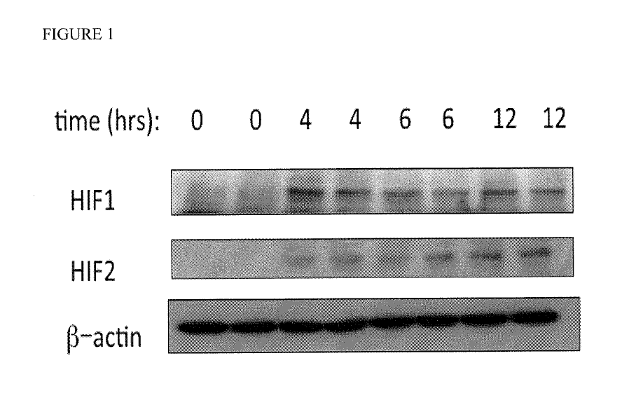

In order to assess the kinetic effects of DMOG on the HIF-1 and HIF-2 protein levels, male C57Bl/6 mice were injected with 8 mg. of DMOG intraperitoneally and colonic crypts were harvested at various time points after injection (FIG. 1). A 8 mg dose results in the elevation of HIF1 and HIF2 proteins 4-12 after DMOG administration as determined by Western blot analysis. There was no observed increase in mortality or GI toxicity at this dose of DMOG observed, nor was there obvious gross morphological changes in the intestine.

### Example 2

### DMOG Protects the Regenerative Capacity of the Colon Following Radiation Injury

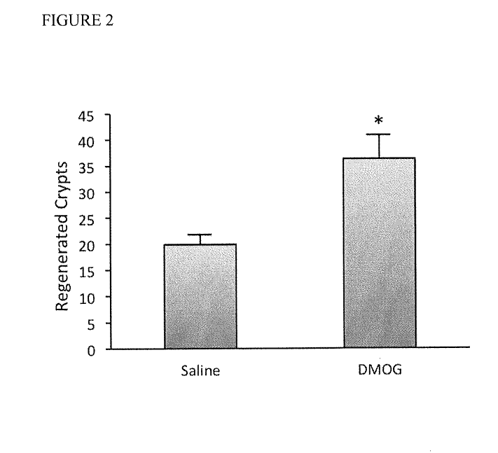

Radio- and chemotoxicity to the GI tract is the result of damage to the intestinal stem cell located within the crypts of Lieberkühn. The intestinal stem cell, defined by its expression of LGR5 is the most rapidly dividing cell in the GI tract and is therefore susceptible to genotoxic damage. In order to assess the effects of DMOG on crypt regeneration after radiation exposure, mice were treated with either saline or 8 mg of DMOG prior to irradiation with a single fraction of 20 Gy to the whole abdomen using a modified TLI jig that shields the upper mouse body. After irradiation, the mice received daily doses of saline or DMOG until they were sacrificed 4.5 days post irradiation. The lower GI tract colon was harvested and subjected to microcolony analysis, which is an in vivo measure of the capacity of intestinal stem cells to regenerate after radiation insult. Remarkably, DMOG treatment exhibited significant radioprotection in the colon, with a 2-fold enrichment in regenerative capacity relative to saline alone (FIG. 2, *p<0.01).

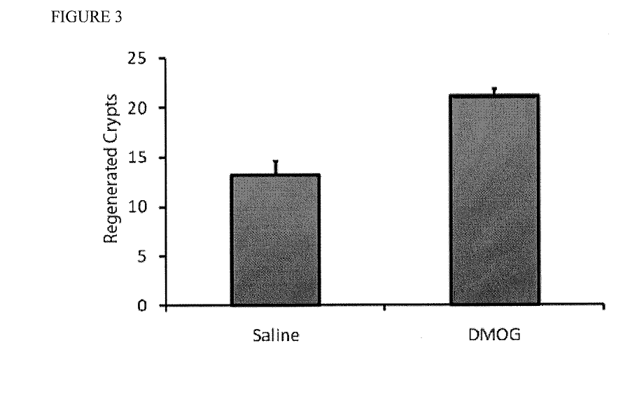

DMOG ameliorates the affects of radiotoxicity even when administered after irradiation. Mice were treated with either saline or 8 mg. of DMOG prior to irradiation with a single fraction of 20 Gy to the whole abdomen using a modified TLI jig that shields the upper mouse body. After irradiation, the mice received daily doses of saline or DMOG until they were sacrificed 4.5 days post irradiation. The lower GI tract colon was harvested and subjected to microcolony analysis, which is an in vivo measure of the capacity of intestinal stem cells to regenerate after radiation insult. Remarkably, DMOG treatment exhibited significant radioprotection in the colon, with a 2-fold enrichment in regenerative capacity relative to saline alone (FIG. 3, *p<0.05 DMOG vs saline).

### Example 3

### DMOG Improves Relative Epithelial Function Following Radiation Injury

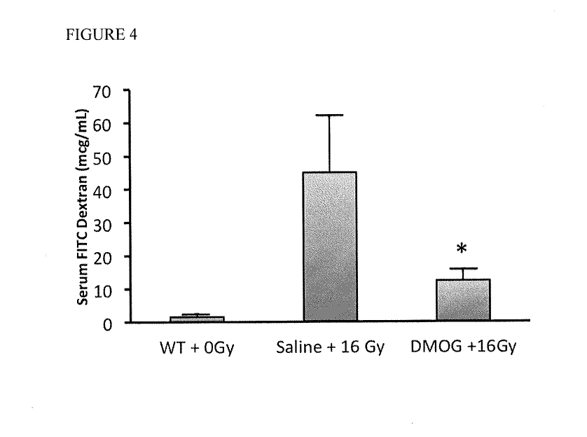

Mortality from GI radiotoxicity results from compromised epithelial integrity manifesting as uncontrolled diarrhea or infections from enteric pathogens due to decreased barrier function. To test whether a PHD inhibitor would increase epithelial integrity after radiation insult, a FITC-dextran assay of the GI tract was performed. FITC-dextran cannot cross the GI epithelia, thus its presence in serum is an indication of compromised epithelial integrity. Following irradiation (20 Gy), mice underwent gavage, with the insertion of 0.6 mg/kg of FITC-dextran (4 kD). Four hours later, levels of FITC-dextran were measured in the blood. Treatment with DMOG results in a 3.5-fold decrease in the presence of FITC-dextran in the serum relative to saline control (FIG. 4). There is almost no uptake in mice that did not receive radiation.

### Example 4

### DMOG Improves Survival Following Radiation Exposure

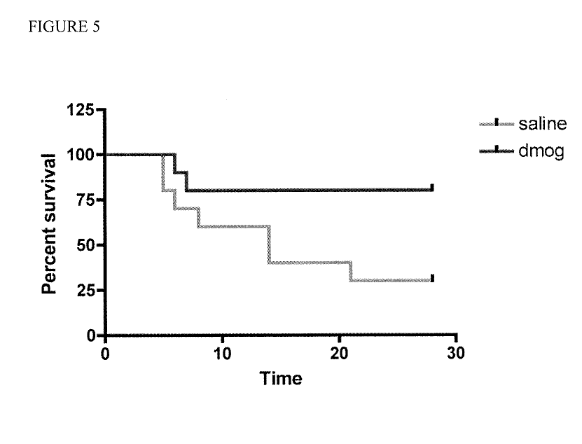

The overall survival of mice receiving 20 Gy of abdominal radiation was dramatically improved by treatment with DMOG (FIG. 5).

What is claimed is:

1. A method of alleviating the affects of lethal radiation dose, the method comprising administering to a subject in need thereof an effective amount of a prolyl hydroxylase domain (PHD) protein inhibitor wherein the PHD inhibitor comprises the formula: 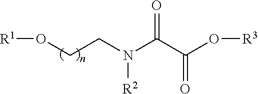
in which
R¹ and R³ are independently members selected from substituted or unsubstituted C₁-C₆ alkyl;
R² is a member selected from H and substituted or unsubstituted C₁-C₆ alkyl;
and n is an integer from 0 to 10.

2. The method of claim 1 wherein the PHD protein inhibitor is administered prior to lethal radiation exposure.

3. The method of claim 1 wherein the PHD protein inhibitor is administered during or after lethal radiation exposure.

4. A method of alleviating proctopathy, the method comprising administering to a subject in need thereof an effective amount of a PHD protein inhibitor.

5. A method of alleviating the affects of abdominopelvic radiation therapy, the method comprising administering to a subject in need thereof an effective amount of a PHD protein inhibitor.

6. The method of claim 1 wherein the PHD protein inhibitor alleviates gastrointestinal toxicity.

7. The method of claim 1 wherein the PHD protein inhibitor alleviates bone marrow toxicity.

8. The method of claim 1 wherein the PHD inhibitor alleviates esophageal toxicity.

9. The method of claim 5 wherein the PHD inhibitor is dimethyloxalyl glycine (DMOG).

10. The method of claim 1 wherein the subject is a mammal.

11. The method of claim 10 wherein the mammal is a human.

12. The method of claim 1 wherein the PHD inhibitor is administered intraperitoneally.
